(19) United States
(12) Patent Application Publication
(10) Pub. No.: US 2023/0310224 A1
(43) Pub. Date: Oct. 5, 2023

(54) Ear Ring Hole Cotton Swab

(71) Applicant: Kevin Dewayne Brister, McComb, MS (US)

(72) Inventor: Kevin Dewayne Brister, McComb, MS (US)

(21) Appl. No.: 17/644,493

(22) Filed: Dec. 15, 2021

(51) Int. Cl. A61F 13/38

(52) U.S. Cl. CPC A61F 13/38

(57) ABSTRACT

A hollow cylindrical body that is made of flexible plastic; that is about two inches in length; the tips that are much smaller in diameter; runs through the body; which sticks out about a half of an inch on both sides.

### TECHNICAL FIELD

The present invention generally relates to a cotton swab that has been improved to better cleanse the hole of a piercing.

### BACKGROUND ART

There is known, a cotton swab in which a tubular tube that has a ball of cotton attached to each end is used to attempt to clean piercings. However this method does not clean the piercing thoroughly. This can cause the inside of the piercing to become infected.

### SUMMARY OF THE INVENTION

To facilitate one's comprehension and understanding of the present invention, that idea must be described not only as it exists as an answer to a subsistent problem, but also as functions physically. Incorporated into this overview is a description of the invention's physical appearance.

In accordance with the present invention, we will describe the basic overall features, shape and design of the Ear Ring Hole Cotton Swab. Materials of which the invention would be produced will also be described.

According to the present invention, the design of the Ear Ring Hole Cotton Swab calls for cotton, plastic and adhesive to be utilized as its major components.

In accordance with the present invention, the Ea Ring. Hole Cotton Swab is a modified cotton swab that is specifically to make it easier for people to thoroughly clean the holes in pierced ears. In order to prevent infection, consumers are encouraged to thoroughly clean the piercing with alcohol. Most people do this by dipping a cotton swab, or paper towel, or tissue in alcohol and then running the swab, or tissue, over the hole. Unfortunately, this system is not very effective because it doesn't clean the interior potion of the piercing.

According to the present invention, the Ear Ring Hole Cotton Swab would be designed with an extremely small swab diameter so that it could be inserted into the piercing for a more thorough, and effective cleaning. According to the present invention, the cotton swab would measure three inches in length and be roughly the size of a regular cotton swab. The center potion of each swab would be made of flexible, hollow plastic tube that was the same diameter of a standard cotton swab. This will ensure that the user could easily grip, and maneuver, the cotton swab.

In accordance with the present invention, each tip of the cotton swab would also be made of flexible plastic, but the tip would be much narrower than the center portion being roughly the diameter of a standard-sized sewing needle. The tips would be wrapped tightly with cotton. Most cotton swabs are made with 100 percent pure cotton that has been sterilized for safety and the Ear Ring. Hole Cotton Swab would be no exception. The cotton would be secured to the plastic tips by means of a simple adhesive, such as glue, if necessary. When completely wrapped in cotton, the tips of the Ear Ring Hole Cotton Swab should be small enough to be easily inserted into the piercing,

According to the present invention, the Ear Ring Hole Cotton Swab would be sold in boxes of 300. The cotton swab could be used to clean all types of body piercings and would not be limited to use only for pierced ears. To use the Ear Ring Hole Cotton Swab, the consumer would simply soak the tip in rubbing alcohol and then insert the swab into the hole of the piercing.

### Proposed Modification

In order to fully appreciate the benefits and the need of an innovation, one should consider any possible modification that would arise from the use of the idea.

I, therefore, shall suggest other application of the Ear Ring Hole Cotton Swab that would merit future consideration and study. The scope of this review may include the use of materials other than those specified by me. In addition, the review may support the use of additional color schemes, diversified application, broader consumer outlets or other possible shape and design alterations that may enhance the utility and salability of my invention. All in all, we shall attempt to identify any variations that would further define the scope of my idea.

One must remember, however, that these modifications suggestions are limited within the latitude of the invention. Some invention lends themselves easily to future application, i.e. different shapes, materials, color schemes, etc. Other inventions, however, are geared to a specific group of consumers, to particular design or to a strict manufacturing process. As a result, any suggestion modification to the invention may be limited within the invention's scope.

The following modifications are suggested to augment the usefulness and popularity for the Ear Ring Hole Cotton Swab. It should not be noted that these and/or other suggested modifications or variations of my invention will not be included in my preliminary patent art.

In accordance with the present invention, the Ear Ring Hole Cotton Swab could come pre-soaked in alcohol. The swab can be packaged in individual airtight wraps. The user would simply remove the swab from the wrap and it would be ready to use. This would make it easier for consumers to carry the product when they traveled or just to keep some on them for use throughout the day.

The product could be sold in variety of different-sized packages. A package of 300 might be too large for some people's needs, so they could also be sold in travel-size packages of 20 or packages of 50 or 100. The product could be packaged in a smaller set of 5 that would be provided to clients by places of business which performed body piercing.

Some cotton swabs are manufactured using tightly wrapped paper instead of plastic. Prototype testing could determine whether plastic, or paper, would be the best choice for use in manufacturing of the Ear Ring Hole Cotton Swab.

In addition, detailed diagrams and technical specifications remain to be created. Nonetheless, there is a clear idea of my intent and goals in submitting my design.

### BRIEF DESCRIPTION OF THE DRAWINGS

FIG. 1 is an overhead view illustrating one preferred embodiment of a cotton swab according to the present invention;

FIG. 2 is an inside perspective view of an example of the cotton swab.

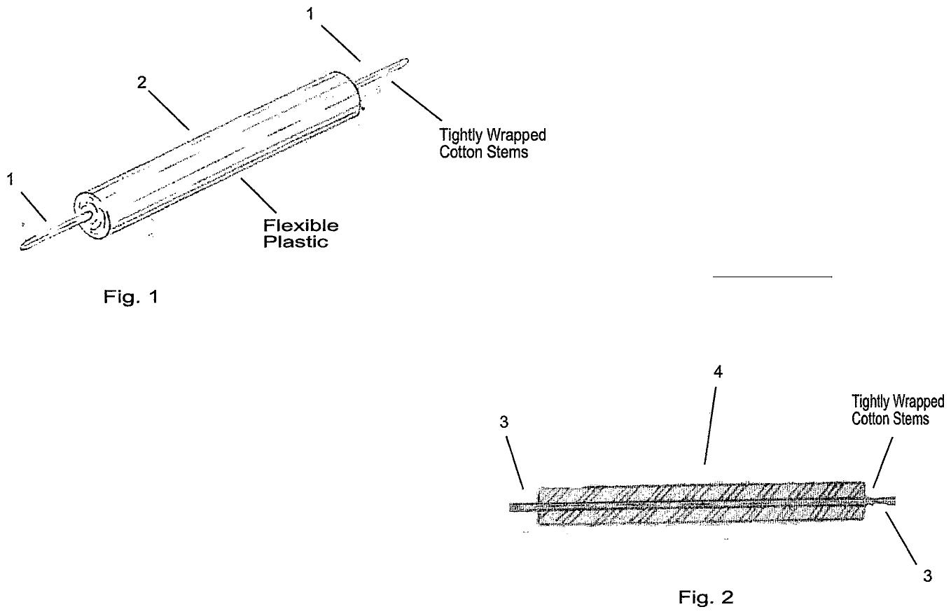

### DETAILED DESCRIPTION OF THE INVENTION

The inventor provides a unique cotton swab for cleansing ear ring piercings. The present invention is described in enabling details in the following examples, which may represent more than one embodiment of the present invention.

According to the embodiment of the present invention 2 is a flexible hollow plastic that would allow the user to grab and hold the cotton swab. In this example the tips 1 are flexible plastic that would stick out of both ends of the present invention. According to the present invention, the tips will stick out approximately a half of an inch on both sides.

An embodiment of the present invention (FIG. 2) the tips 3, are glued and wrapped tightly with 100 percent cotton. In an example 4, is illustrating how the tip's flexible plastic runs through the center of 2 the hollow plastic that will be sealed for a stronger hold.

It will be apparent to one with skill in the art of the Ear Ring Hole Cotton Swab; the invention may be provided some or all of the mention features and components without departing from the spirit and scope of the present invention. It will also be apparent to the skill artisan that the embodiments described above are specific examples of a single broader invention which may have greater scope than any of the singular description taught. There may be many alterations made in the description without departing from the spirit and scope of the present invention.

1. A cotton swab comprising:
A hollow cylindrical body (2) that is made of flexible plastic; that is about two inches in length; the tips (1) that are much smaller in diameter; runs through the body (2); which sticks out about a half of an inch on both sides.

2. The tips (3) the cotton swab are about a half of an inch in length; which would have 100 percent cotton glued and wrapped tightly around the tips; a small enough diameter to fit inside of an ear ring hole piercings.
